(19) United States
(12) Patent Application Publication
(10) Pub. No.: US 2021/0079458 A1
(43) Pub. Date: Mar. 18, 2021

(54) AMPLIFICATION METHOD OF A SINGLE STRANDED DNA

(71) Applicants: National Guard Health Affairs, Riyadh (SA); King Saud bin Abdulaziz University for Health Sciences, Riyadh (SA); King Abdullah International Medical Research Center, Riyadh (SA)

(72) Inventors: Atef Nehdi, Riyadh (SA); Mohamed Boudjelal, Riyadh (SA); Nosaibah Samman, Riyadh (SA)

(73) Assignees: National Guard Health Affairs, Riyadh (SA); King Saud bin Abdulaziz University for Health Sciences, Riyadh (SA); King Abdullah International Medical Research Center, Riyadh (SA)

(21) Appl. No.: 16/569,081

(22) Filed: Sep. 12, 2019

(51) Int. Cl. C12Q 1/6853

(52) U.S. Cl. CPC C12Q 1/6853

(57) ABSTRACT

A method for preparing a single stranded DNA by the polymerase chain reaction in high yield and purity. The method employ one regular primer and one modified primer at the 5′-end. The modified primer has a primer segment linked to an oligo- or polynucleotide segment through a 5′-5′-phosphodiester bond. Using the modified primer, the PCR product includes two complementary DNA strands having different lengths and separable from one another.

### BACKGROUND OF THE INVENTION

### Field of the Disclosure

The present invention relates to a nucleic acid amplification method by polymerase chain reaction (PCR) utilizing one modified primer attached at the 5′-end to an oligo or polynucleotide through a 5′-5′-phosphodiester bond.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present disclosure.

Polymerase Chain Reaction (PCR) is likely the most widely used method in modern molecular biology and biotechnology, and is currently in use in genetic testing, diagnostics, forensics, and biodefense [Kolmodin et al. “Nucleic Acid Protocols Handbook” pp 569-580 (Rapley, R. ed., Humana Press 2000); Budowle et al. Science 301, 1852-1853 (2003); Sato et al. Legal Medicine, 5 (Suppl. 1), S191-S193 (2003); Saldanha et al. J. Medical Virol. 43, 72-76 (1994); Dahiya et al., Biochemistry and Molecular Biology International, 44, 407-415 (1998); and Elnifro et al. Clin. Microbiol. Rev. 13, 559-570 (2000)]. PCR is described in U.S. Pat. Nos. 4,683,195 and 4,683,202—each of which incorporated by reference in their entirety. In each cycle of the PCR amplification process, there are typically several steps described herein in details below. Generally, the double-stranded DNA target sequence is first thermally denatured at elevated temperatures at about 95° C. The denatured DNA is annealed to a forward and a reverse oligonucleotide primer to each DNA strand at lower temperatures that depend on the length of the primer usually 2-3° C. below the melting temperature of the template/primer. The forward and reverse oriented oligonucleotide primers are then each extended from their 3′-termini at an elevated temperature at about 70° C. by thermally stable, magnesium ion-dependent, DNA polymerase which incorporates 2′-deoxyribonucleoside 5′-triphosphates (dNTPs) at the 3′-end sequentially of each primer

Modified PCR methods, known as asymmetric PCR, have been developed for the synthesis of a single strand DNA. Asymmetric PCR methods have low product yields. The low yield of the methods is due to the fact that the newly generated ssDNA competes with the forward primer, which is in large excess in the reaction mixture, blocking its binding to the DNA template and thereby, terminating the amplification reaction (see FIG. 1). During the initial cycles of PCR the forward primer has a concentration advantage in binding to the DNA template, but as the number of PCR cycles increases the concentration of the amplified ssDNA complementary to the template is increased. As the concentration of the amplified ssDNA increases, the ssDNA becomes more competitive in binding to the template because it is longer in length than the primer. The competition appears early in the amplification reaction and the binding advantage to the template is shifted to favor of the amplified single ssDNA and thereby the amplification reaction is terminated.

U.S. Pat. No. 5,627,054A-incorporated herein by reference in its entirety discloses a PCR method for determining both the presence and the number of copies of a target nucleic acid sequence in a sample being analyzed. The method comprises two steps. The first step is a symmetric amplification of a double stranded nucleic acid followed by asymmetrically amplification of the double stranded nucleic acid. The asymmetric amplification comprises adding a first primer to be extended and a second non-extendable primer, named competitor primer, and thereby preventing one of the strands from being amplified. Two types competitor of primers are disclosed. The first is 20-50 nucleotides in length with 5′-portion complementary the amplification primer binding site on the selected strand and 3′-noncomplementary portion that cannot be annealed to the selected strand, and thus cannot be extended. The second competitor primer with a sequence that is fully complementary to the amplification primer binding site on the selected strand with modified 3′-nucleotide terminus that cannot be extended. Example of modified 3′-nucleotides which cannot be extended include dideoxynucleotide or a protecting group at the 3′-OH.

U.S. Pat. No. 6,958,217B2 discloses a method of isolating single stranded polynucleotide tags. The method comprises providing a double stranded nucleic acid, cleaving one strand by an agent that recognizes a double stranded nucleic acid, and isolating the single stranded nucleic acid. Once the single stranded DNA tag is isolated, it may be sequenced, quantified, or hybridized or ligated to another nucleic acid sequence. U.S. Pat. No. 6,958,217B2 does not disclose any efficient method for amplifying single stranded DNA by PCR.

U.S. Pat. No. 8,133,669B2 discloses a PCR method utilizing a modified nucleotide triphosphates modified at the 3′-position with thermolabile protecting group, template, and a thermostable enzyme. At ambient temperature where PCR mixtures are prepared, non-specific priming of the template is possible due to the low stringency conditions leading to the amplification of undesired DNA. U.S. Pat. No. 8,133,669B2 disclosure overcome the problem by utilizing mucleotide triphosphate wherein the 3′-hydroxyl is protected by a thermolabile group. The presence of the thermolabile group minimizes the polymerization at low temperature and hence the amplification of undesired DNA. As the temperature rises to about 95° C., the thremolabile protecting groups are removed allowing the polymerization reaction to proceed.

CN1475576A discloses a method for preparing single stranded DNA comprising anchoring a primer on micro-particles, i.e., a solid support, providing a template, nucleotide triphosphate, and DNA-polymerase to obtain a double stranded DNA immobilized on solid support. Heat denaturation of the produced double stranded DNA lead to the separation of the two strands as one strand is released into the solution whereas the other is bound to the solid support. Separating the solid support from the solution leads to the separation of the product strands. The method requires the preparation of immobilized primers on solid support which is laborious and may hinder priming long strand of DNA.

None of the above references discloses an efficient method for the amplification of a single stranded DNA. It is therefore, one of the objectives of the invention is to disclose an efficient method for obtaining a single stranded DNA in high yield.

The foregoing paragraphs have been provided by way of general introduction, and are not intended to limit the scope of the following claims. The described embodiments, together with further advantages, will be best understood by reference to the following detailed description taken in conjunction with the accompanying drawings.

### SUMMARY

One aspect of the invention is directed to a PCR method for the synthesis of a single stranded nucleic acid sequence comprises:

preparing a polymerase chain reaction comprising a template, a first modified primer, a second primer, nucleotide triphosphates, and a DNA-polymerase, and

carrying out amplification reactions by cycling the temperature of the reaction mixture between 20° C. and 100° C. to produce a product DNA strands,

separating the produced DNA strands;

wherein the modified primer comprises a first segment of 10-50 nucleotides complementary to the template having an extendable 3′-end and a 5′-end linked to the 5′-end of a second oligonucleotides of 10-100 nucleotides or a polynucleotides.

In a preferred embodiment, the product DNA strands are separated by denaturing electrophoresis.

In another preferred embodiment, the template is a DNA strand.

In another preferred embodiment, the DNA-polymerase is a thermophilic DNA-dependent DNA-polymerase.

In another preferred embodiment, the second segment is oligo-dA, or poly-dA.

In another preferred embodiment, the product DNA strands are removed from the reaction mixture by binding to a solid support modified by a fragment of oligo- or poly(dT) or poly(U).

In another preferred embodiment, the amplified DNA is released from the solid support by denaturation.

In another preferred embodiment, the template is a RNA strand.

In another preferred embodiment, the DNA-polymerase is RNA-dependent DNA-polymerase.

Another aspect of the invention is directed to a PCR method for the synthesis of a single stranded nucleic acid sequence comprises:

preparing a polymerase chain reaction comprising a template, a modified primer, a reverse primer, nucleotide triphosphates, and a nucleic acid-polymerase,

carrying out amplification reactions by cycling the temperature of the reaction mixture between 20° C. and 100° C. to produce a product DNA strands, and

separating the resulting DNA strands;

wherein the modified primer comprises a first segment of 10-50 nucleotides complementary to the template having an extendable 3′-end and a 5′-end linked to the 5′-end of at least one nucleotide which is linked through its 3′-end to a 3′-end of a second segment of oligonucleotid having 10-100 nucleotides or a polynucleotides.

In a preferred embodiment, the produced DNA strands are separated by denaturing electrophoresis.

In another preferred embodiment, the template is a DNA strand.

In another preferred embodiment, the DNA polymerase is a thermophilic DNA-dependent DNA polymerase.

In another preferred embodiment, the second segment is oligo-dA, or poly-dA.

In another preferred embodiment, the product DNA strands are removed from the reaction mixture by binding to a solid support modified by a fragment of oligo- or poly(T) or (U).

In another preferred embodiment, the produced DNA strands are separated from the solid support by denaturation.

In another preferred embodiment, the template is a RNA strand.

In another preferred embodiment, the DNA polymerase is RNA-dependent DNA polymerase.

Another aspect of the invention is directed to a polynucleotide primer comprising a first segment and a second segment linked by 5′-5′-phosphodiester bond wherein the first segment comprises 10-50 nucleotides complementary to a target nucleic acid for amplification and the second segment is an oligonucleotide of 10-100 residues or a polynucleotide.

In a preferred embodiment, the second segment is oligo- or poly-dA, oligo- or poly-dT, oligo- or poly-dG, or oligo- or poly-dC.

Another aspect of the invention is directed to a first nucleic acid sequence complementary to a desired single stranded DNA linked to a second oligo- or polynucleotide through 5′-5′-phosphodiester bond.

In a preferred embodiment, the second oligo- or polynucleotide is oligo- or poly-dA, oligo- or poly-dT, oligo- or poly-dG, or oligo- or poly-dC.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 shows a schematic diagram showing the premature termination of conventional ssDNA amplification method after few PCR cycles.

FIG. 2A shows a simplified schematic representation of a nucleotide with 3′ and 5′ termini.

FIG. 2B shows a schematic diagram of one embodiment of a modified primer of the invention and comprising 5′-5′ phosphodiester linkage.

FIG. 3A shows a simplified schematic representation of a nucleotide with 3′ and 5′ termini.

FIG. 3B shows a schematic diagram of an inverted nucleotide with a 3′-3′ and 5′-5′ phosphodiester linkages.

FIG. 4 shows a schematic diagram of the template directed ligation method to generate 5′-5′-phosphodiester linkage.

FIG. 5 shows the DNA-polymerase catalyzed polymerization on a template comprising the 5′-5′-linkage which terminates the polymerization reaction.

FIG. 6 shows the DNA-polymerase catalyzed polymerization on a template comprising the 3′-3′- and 5′-5′-linkages which terminates the polymerization reaction.

FIG. 7 is a schematic representation of the PCR method of the invention using a modified primer comprising a 5′-5′-phosphodiester bond. In set to the right shows the separation of the two strands produced by PCR.

FIG. 8 is schematic representation of the PCR method of the invention using a modified primer comprising a single inverted nucleotide linking the template with oligo- or polynucleotide sequence. The modified primer contains 3′-3′- and 5′-5′-phosphodiester bonds. In set to the right shows the separation of the two strands produced by PCR.

FIG. 9 shows the mobility of various PCR products on 4% agarose gel and denaturing gel.

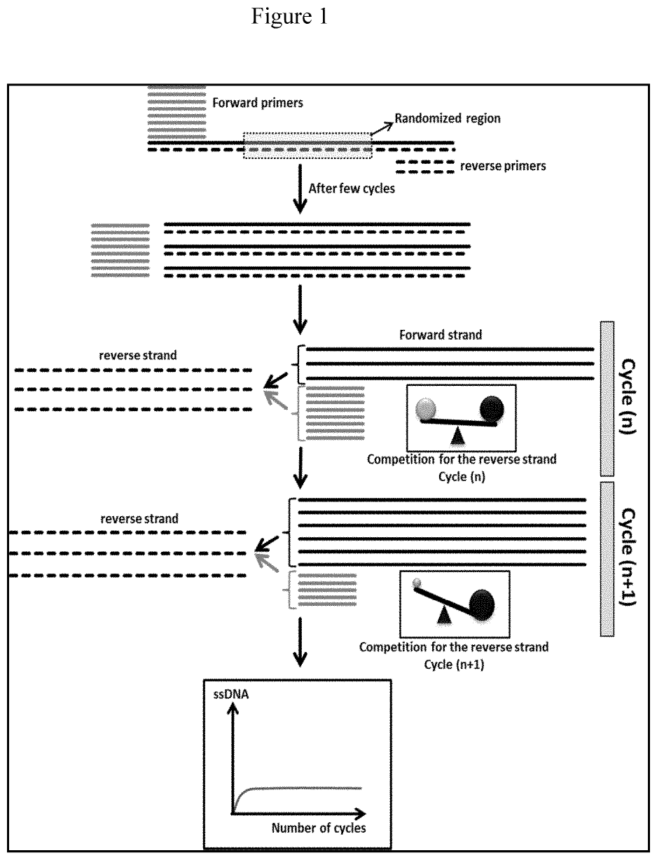

### DETAILED DESCRIPTION

Embodiments of the present disclosure will now be described more fully hereinafter with reference to the accompanying drawings, in which some, but not all embodiments of the disclosure are shown. The present disclosure will be better understood with reference to the following definitions.

All publications mentioned herein are incorporated herein by reference in full for the purpose of describing and disclosing the methodologies, which are described in the publications, which might be used in connection with the description herein. Nothing herein is to be construed as an admission that the inventors are not entitled to antedate such disclosure by virtue of prior disclosure. Also, the use of “or” means “and/or” unless stated otherwise. Similarly, “comprise,” “comprises,” “comprising” “include,” “includes,” and “including” are interchangeable and not intended to be limiting.

As used herein, the term “compound” is intended to refer to a chemical entity, whether in a solid, liquid or gaseous phase, and whether in a crude mixture or purified and isolated.

As used herein, the term “salt” refers to derivatives of the disclosed compounds, monomers or polymers wherein the parent compound is modified by making acid or base salts thereof. Exemplary salts include, but are not limited to, mineral or organic acid salts of basic groups such as amines, and alkali or organic salts of acidic groups such as carboxylic acids. The salts of the present disclosure can be synthesized from the parent compound that contains a basic or acidic moiety by conventional chemical methods. Generally such salts can be prepared by reacting the free acid or base forms of these compounds with a stoichiometric amount of the appropriate base or acid in water or in an organic solvent, or in a mixture of the two; generally non-aqueous media like ether, ethyl acetate, ethanol, isopropanol, or acetonitrile are preferred. As used herein, the term “about” refers to an approximate number within 20% of a stated value, preferably within 15% of a stated value, more preferably within 10% of a stated value, and most preferably within 5% of a stated value. For example, if a stated value is about 8.0, the value may vary in the range of 8±1.6, ±1.0, ±0.8, ±0.5, ±0.4, ±0.3, ±0.2, or ±0.1.

The method of the invention is directed to a method of amplification and purification of a single stranded DNA by polymerase chain reaction (PCR) in high purity and yield. The PCR method for the amplification of nucleic acid sequences is a well-known method in the art and is described in detail below for the amplification of DNA from a DNA or RNA template (see for example Promega—https.//www.promega.com/resources/product-guides-and-selectors/protocols-and-applications-guide/pcr-amplification/-incorporated herein by reference in its entirety). All reagents and equipment required to carry out standard PCR amplification including, but not limited to, thermocycler, thermostable DNA-polymerases, deoxynucleotide triphosphates, and buffers are commercially available.

As used herein, the term “amplification” refers to any in vitro method for increasing the number of copies of a nucleotide sequence with the use of a template-dependent DNA-polymerase. Nucleic acid amplification results in the incorporation of nucleotides into a DNA primer thereby forming a new molecule complementary to a target template. The newly formed nucleic acid molecules and the original template can be used as templates in subsequent cycles to synthesize additional copies of the nucleic acid. As used herein, one amplification reaction may consist of many rounds of replication. One PCR reaction may consist of a number of cycles in the range of at least 5, preferably 10, preferably 20, preferably 30, preferably 40, preferably 50, preferably 100, preferably 150, preferably 200, preferably 250, preferably 300 or more cycles of denaturation and synthesis of a DNA molecule. In general, a PCR reaction mixture comprises a forward primer, a reverse primer, a template, a nucleotide polymerase, nucleotide triphosphate, magnesium chloride, potassium chloride, a buffer at pH in the range of 7.0-9.5, preferably 7.5-9.0, preferably 8.0-8.6, preferably about 8.4.

As used herein, the term template refers to a target nucleic acid to be amplified. The target nucleic acid to be amplified by the method of the invention is generally considered to be any nucleic acid or nucleic acid analog capable of being amplified by PCR methods known in the art. By way of example, target nucleic acids specifically contemplated in the context of the disclosure, may include, but are not limited to genomic DNA, cDNA, RNA, mRNA, cosmid DNA, BAC DNA, PAC DNA, YAC DNA, and synthetic DNA. In a contemplated embodiment, genomic DNA is from a prokaryotic or eukaryotic cell or tissue and utilized as the sample DNA in the disclosed DNA amplification method. In other embodiments, mRNA tail is isolated and amplified directly to DNA by the PCR method of the invention. Alternatively, the mRNA may be reverse transcribed by a reverse transcriptase to obtain cDNA, which is then used as the sample DNA for DNA amplification using the presently disclosed method. In other contemplated embodiments, cDNA may be obtained and used as the sample DNA to be amplified. In some embodiments, the template is a DNA or RNA template present in an amount in the range of 10 to 1,000,000 copies/100 mL, preferably 50-500,000 copies/100 mL, preferably 100-100,000 copies/100 mL in a PCR reaction mixture. In some preferred embodiments, the concentration of the template is in the range of 1-20 ng/μL, preferably 2-15 ng/μL, or preferably 5-10 ng/μL.

As used herein, the term “primer” refers to a short sequence of DNA or RNA which is complementary to the 5′-end of a target DNA or RNA. The primer should be sufficiently long to provide sufficient specificity for binding to the target 5′-terminus of a template and minimize non-specific binding to the target nucleic acid. It is contemplated that the primer is at least 10, preferably 15, preferably 25, preferably 30, preferably 35, preferably 40, preferably 45, preferably 50, preferably 60, preferably 70, preferably 80, preferably 90, preferably 100 nucleotides in length. Accordingly, nucleotide sequences may be selected for their ability to selectively form duplex molecules with complementary stretches of genes, DNA, or RNAs, or more specifically to provide primers for amplification of DNA preparations from DNA or RNA directly or indirectly derived from cells, cell lysates, viruses including retroviruses, and tissues. Primers of the present disclosure are used to amplify single stranded DNA from a target nucleic acid. The terms “forward primer” and “reverse primer” refer to primers that would amplify one strand from the 5′-end to the 3′-end direction, whereas the reverse primer amplify the complementary strand from the 5′-end to the 3′-end direction. The terms “forward primer” and “reverse primers” are used for convenience to distinguish one from the other, but they are functionally equivalent unless otherwise indicated. Each of the primers is present in the PCR medium at concentration in the range 1.0-0.05 mM, preferably 0.6-0.10 mM, preferably 0.3-0.2 mM, preferably about 0.25 mM.

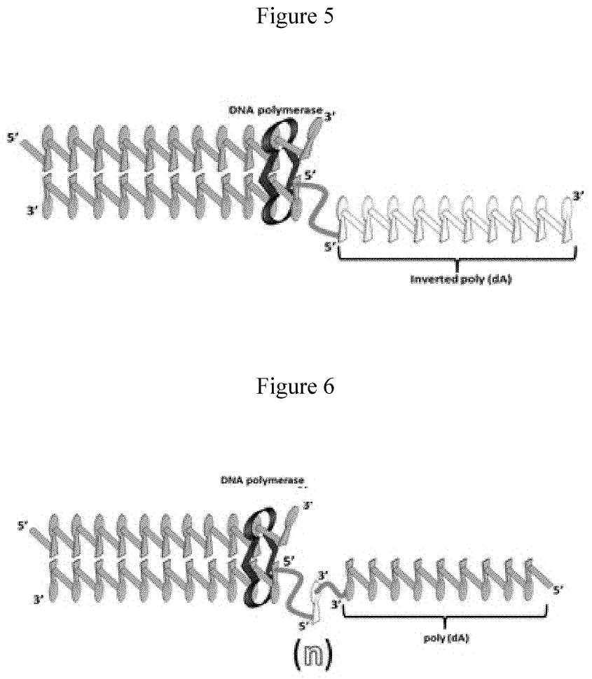

In conventional PCR mixture, two primers are used complementary to the 5′-ends of the target nucleic acid and its complementary sequence. Consequently, the PCR product consists of two complementary strands of the same length and approximately the same molecular weight. Thus, the separation of the two strands can be challenging by conventional methods. The PCR method of the invention obviates the problem by allowing the separation of the two strands. The method of the invention relies on the fact that DNA-polymerases require a free 3′-end of a primer for a polymerization chain reaction to grow the primer from the 3′-end to the 5′-end. The PCR amplification method of the invention utilizes one regular primer and one modified primer linked at its 5′-end to an oligo or polynucleotide through a 5′-5′-phosphodiester linkage. After the initial cycle of PCR, a new template is formed linked to an oligo- or poly-nucleotide through a 5′-5′-linkage. In subsequent cycles, the DNA polymerase will synthesize a complementary strand of the new template until it reaches the 5′-5′linkage, which terminates the polymerization reaction because the direction of the template nucleic acid chain is reversed (see FIGS. 5 and 6).

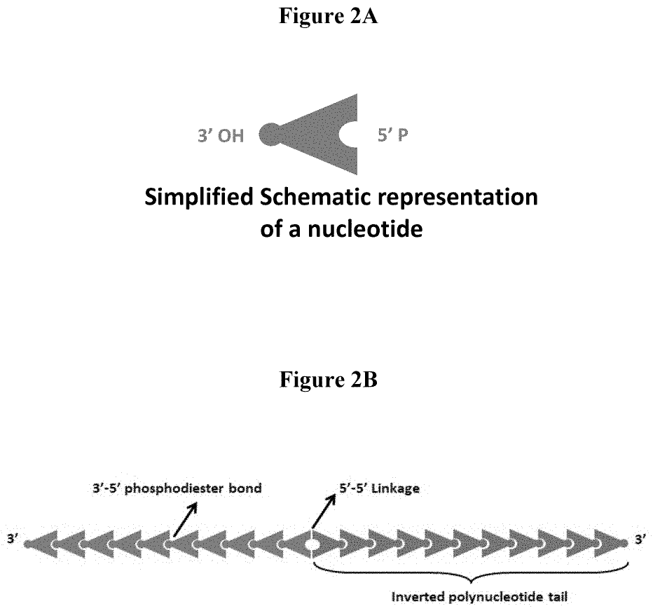

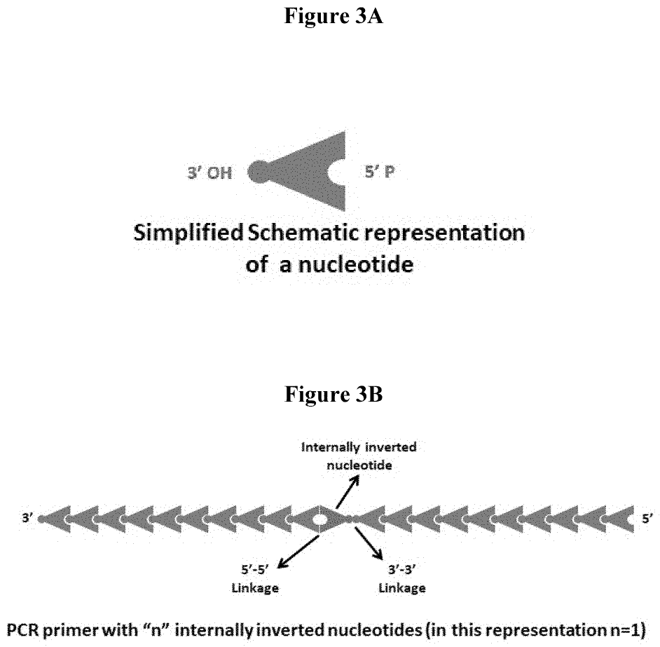

One aspect of the invention is directed to a modified primer comprising a first segment complementary to the 5′-end of the template linked at the 5′-end to the 5′-end of a second nucleic acid segment forming a 5′-5′ linkage. As used herein, an “inversion linkage” refers to a phosphodiester linkage which joins the backbone of one portion of a polynucleotide to the backbone of an adjacent portion of the same polynucleotide having an opposite orientation. The term particularly embraces 5′-5′ and 3′-3′ linkages in conventional nucleic acids nomenclature. In one embodiment, the modified primer comprises a first segment complementary to the target nucleic acid sequence of 10-50 nucleotides in length linked at the 5′-end to the 5′-end of a second nucleotide segment through a 5′-5′-linkage phosphodiester bond of an oligonucleotide or polynucleotide having at least 2, preferably 5, preferably 10, preferably 30, preferably 50, preferably 100, preferably 200, preferably 300, preferably 400, preferably 500 or more nucleotides (see FIGS. 2A and 2B). In some other embodiments, the modified primer comprises a first segment complementary to the target nucleic acid sequence of 10-50 nucleotides in length linked at the 5′-end to the 5′-end of an inverted nucleotide or an oligonucleotide of 2-6 nucleotides through a phosphodiester bond (5′-5′-linkage) and the 3′-end of the one nucleotide or oligonucleotides is linked at the 3′-end to the 3′-end (3′-3′-linkage) of a second segment of an oligonucleotides or polynucleotides having at least 1, preferably 2, preferably 5, preferably 10, preferably 30, preferably 50, preferably 100, preferably 200, preferably 300, preferably 400, preferably 500 or more nucleotides (see FIGS. 3A and 3B).

Primer oligonucleotides or polynucleotides containing 3′-3′- or 5′-5′-phosphodiester units can be synthesized by methods well-known to those skilled in the art and are described, for example, in M. Koga et al., J. Org. Chem. 56:3757, 1991, EP 0 464 638, US20040005595, and EP 0 593 901, U.S. Pat. No. 5,750,669—each of which is incorporated herein by reference in its entirety. Properly protected 3′- and 5′-phosphimidate derivatives for use in automated nucleic acid synthesizer for making 5′-5′ and 3′-3′ linkages can be synthesized by well-known methods in the art such as those described U.S. Pat. No. 5,750,669—incorporated herein by reference in its entirety. Alternatively, they can be obtained from a commercial source such as Glen Research (www-glenresearch.com). In particular, Glen Research provides dT-5′-CE (i.e., 3′-dimethoxytrityl-2′-deoxythymidine 5′-[(2-cyanoethyl)-(N,N-diisopropyl)]-phosphoramidite), dA-5′-CE, dC-5′-CE, and dG-5′-CE phosphoramidites for the synthesis of 3′-3′- and 5′-5′-phosphodiester linkages.

The modified primer of the invention may be synthesized using a procedure such as that described in US2004/0005595-incorporated herein by reference in its entirety. For example, the primer shown schematically in FIGS. 2A and 2B may be synthesized from either end on an automated DNA synthesizer. Starting with a 3′-derivatized controlled pore glass (CPG) columns and 3′-phosphoramidites, an oligonucleotide can be synthesized in the 3′ to the 5′ direction forming a first segment containing only 3′-5′-phosphodiester linkages. When the first segment is completed, at least one 5′-phosphoramidites are coupled to the nascent chain forming one 5′-5′ phosphodiester linkage followed by growing the chain in 5′ to 3′ direction until the second segment is completed to produce a template having the structure shown in FIGS. 2A and 2B. Similarly, a template having the structure shown schematically in FIGS. 3A and 3B may be synthesized from either direction. It could start with a 3′-derivatized CPG column and 3′-phosphoramidites to grow the nucleic acid chain in the 3′ to 5′ direction to form a first segment comprising 3′-5′ phosphodiester bonds. When the first segment is completed, at least one 5′-phosphoramidite is coupled to the nascent chain forming 5′-5′ phosphodiester bond with protected 3′-hydroxyl group protected. Deprotecting the 3′-hydroxyl group and coupling to 3′-phosphoramidites produces 3′-3′-phosphodiester bond and a protected 5′-hydroxyl group which can be extend as much as desired. The modified primer may be synthesized from the opposite direction starting with 5′-derivatized CPG columns and 3′-phosphoramidites followed by similar producers as described above.

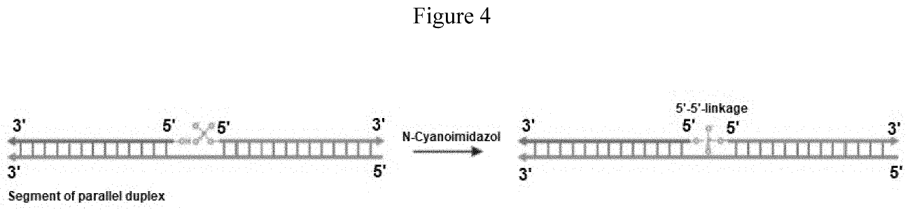

The modified primers of the invention may also be prepared by template-directed chemical ligation method to obtain 3′-3′ and 5′-5′-phosphodiester bond using N-cyanoimidazol as coupling reagent [see for example Chen et al. (2014) 4, article number 4595 and US20050208503—each of which incorporated herein by reference in its entirety]. In the method, two nucleic acid segments complementary to a template are brought together on a nucleic acid template. One of the segment forms a parallel duplex with the template with the template, whereas the other segment is in the normal orientation bringing a free 5′-phosphate in the vicinity of a free 5′-hydroxyl group (see FIG. 4). The addition of coupling reagent such as N-cyanoimidazol, dicyclohexyldiimide (DCC) or analogs thereof would lead to the formation of new phosphodiester bond between the 5′-phosphate and the 5′-hydroxyl group. A similar procedure would produce a 3′-3′-phosphodiester bond.

While there is no restriction on the number of the nucleotides in the second segment of the modified primer, at least one nucleotide must be attached. Another requirement is that the attached oligo- or polynucleotide should be of sufficient length that would facilitate easy separation of the two complementary strands produced by PCR by well-known methods in the art such as, but not limited to ion exchange chromatography, gel electrophoreses, and capillary electrophoreses. In some embodiment, the attached nucleic acid to the primer segment polynucleotides, preferably homo-polynucleotide such as poly-dA, poly-dT, poly-dG, or poly-dC.

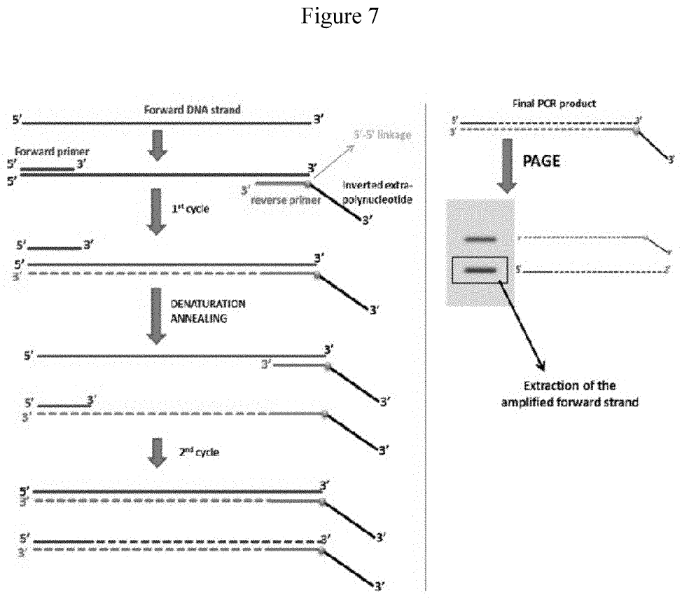

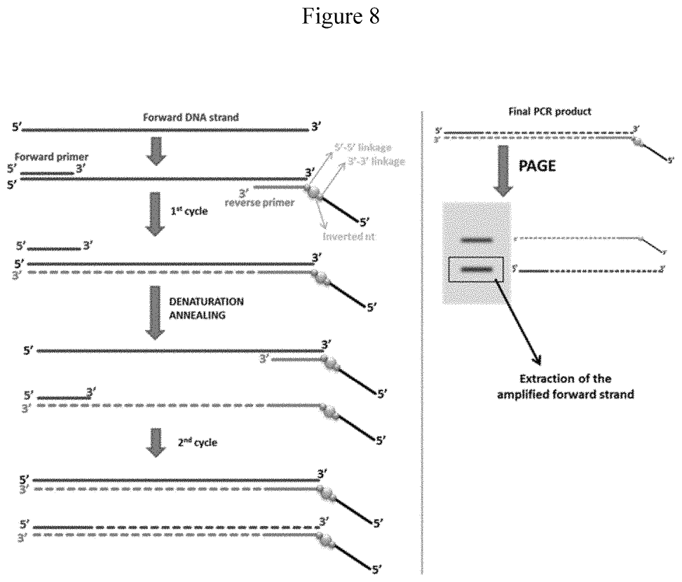

The PCR method of the invention utilizes one modified primer at the 5′-end described herein in one direction and a second regular primer in the opposite direction (see FIGS. 7 and 8), leading to the formation of partly complementary strands having different length which can be separated with relative ease. In some embodiment, the modified reversed primer is attached to an oligo- or polynucleotide through its 5′-end forming a 5′-5′-phosphodiester bond (FIG. 7). The oligo- or polynucleotide sequence can be any sequence which may facilitate the separation of the produced complementary strands from the reaction mixture and one another. In some embodiments, the oligonucleotide attached to the primer through 5′-5′ linkage is at least 1, preferably 2, preferably 5, preferably, 10, preferably 20, preferably 30, preferably 40, preferably 50, preferably 60, preferably 70, preferably 80, preferably 90 nucleotides in length. In some instances, the oligonucleotide is an oligo-dA, oligo-dG, oligo-dT, or oligo-dC. Similarly, the attached polynucleotide attached to the primer through 5′-5′ linkage is at least 100, preferably 150, preferably 200, preferably 300, preferably 400, preferably 500 nucleotides in length. It may be homopolymer such as poly-dA, poly-dG, poly-dT, or poly-dC, or a heteropolymer of any polynucleotide sequence. In some other embodiments, the reverse primer and the oligo- or polynucleotide are attached through a single reverse nucleotide or reverse oligonucleotide sequence of 2, 3, 4, or 5 nucleotides in length in which the 5′-hydroxyl of the single nucleotide or oligonucleotide is bonded to the 5′-end-of the reverse primer through a phosphodiester bond and the 3′-hydroxyl of the reverse single or oligo is bonded to the 3′-end of the oligo- or polynucleotide through a phosphodiester bond (see FIG. 8).

As used herein the phrase “DNA-polymerase” refers to any template directed DNA-polymerase. DNA-polymerases synthesize the formation of a DNA molecule complementary to a single-stranded DNA or RNA template by extending a primer in the 5′-3′-direction. The DNA-polymerase may be a DNA-dependent DNA-polymerase which utilizes a primed DNA-template as substrate and synthesizes the complementary sequence of said template. In some other instances, the DNA polymerase is an RNA-dependent DNA-polymerase, also known as reverse transcriptase, which recognizes a primed-RNA-template as a substrate and catalyzes the formation of a DNA strand complementary to the RNA strand. Examples of DNA-polymerases to be used in the method of invention include, but are not limited to, retroviral reverse transcriptases, retrotransposon reverse transcriptases, hepatitis B reverse transcriptase, cauliflower mosaic virus reverse transcriptase, bacterial reverse transcriptase, Escherichia coli pol-I or a fragment thereof known as the Klenow fragment, Tth DNA polymerase, Taq DNA polymerase [Saiki, R. K., et al, Science 239:487-491 (1988); and U.S. Pat. Nos. 4,889,818 and 4,965,188—each of which incorporated herein by reference in its entirety], Tne DNA polymerase (WO 96/10640 and WO 97/09451—each of which incorporated herein by reference in its entirety), Thermococcus marinus (Tma) DNA polymerase (U.S. Pat. No. 5,374,553 incorporated herein by reference in its entirety) and mutants, variants or derivatives thereof (see, e.g., WO 97/09451 and WO 98/47912—each of which incorporated herein by reference in its entirety). Preferred reverse transcriptases for use in the invention include those that have reduced, substantially reduced or eliminated RNase H activity. RNase H activity refers to an intrinsic enzymatic activity of many known reverse transcriptases which hydrolyses an RNA strand in an RNA-DNA duplex. By an enzyme “substantially reduced in RNase H activity” is meant that the enzyme has less than about 20%, more preferably less than about 15%, 10% or 5%, and most preferably less than about 2%, of the RNase H activity of the corresponding wildtype or RNase H enzyme such as wildtype Moloney Murine Leukemia Virus (M-MLV), Avian Myeloblastosis Virus (AMV) or Rous Sarcoma Virus (RSV) reverse transcriptases. The RNase H activity of any enzyme may be determined by a variety of assays, such as those described, for example, in U.S. Pat. No. 5,244,797, Kotewicz, M. L., et al, Nucl. Acids Res. 16:265 (1988) and Gerard, G. F., et al., FOCUS 14(5):91 (1992)—each of which incorporated herein by reference in its entirety.

In some preferred embodiments, the DNA-polymerase is a thermostable DNA-polymerase usually obtained from thermophilic microorganisms. As used herein “thermostable” refers to a DNA-polymerase which is more resistant to denaturation and inactivation by heat. Mesophilic DNA-polymerases may be denatured and inactivated by heat treatment. For example, the mesophilic T5 DNA-polymerase activity is totally inactivated by exposing the enzyme to a temperature of 90° C. for 30 seconds. As used herein, a thermostable DNA-polymerase activity is more resistant to heat inactivation than a mesophilic DNA-polymerase. However, a thermostable DNA-polymerase does not mean to refer to an enzyme which is totally resistant to heat inactivation, and thus, heat treatment may reduce the DNA polymerase activity to some extent. A DNA-polymerase is considered thermostable if it maintains more than 50% of its activity after being heated at 95° C. for at least 5 min, preferably 10, min, preferably 20 min or more. A thermostable DNA polymerase typically will also have a higher optimum temperature than mesophilic DNA polymerases. Many thermostable DNA-polymerases suitable for PCR are commercially available from laboratory supply vendors such as Sigma-Aldrich [https-://www.sigmaaldrich.com/catalog/product/roche] Theromo-Fisher Scientific, USA [https-://www.thermofisher.com/us/en/home.html], Applied-Biosystem, U.S.A https-://www.fishersci.com/shop/products/applied-biosystems-amplitaq-gold-dna-polymerase-gold-buffer-mgcl-sub-2-sub-6/p-4926873]. Table 1 summarizes some properties of the most, but not all, used thermostable DNA-polymerases in PCR. The choice of the DNA-polymerase is dependent on the end use of the product DNA. For example, Vent, Deep Vent, Pfu, and T th have intrinsic proof reading capability and are able to produce product over 30 kbp long. For example, commercially available Pfu typically results in an error rate of 1 in 1.3 million base pairs and can yield only 2.6% mutated products when amplifying 1 kb fragments using PCR. However, Pfu is slower than other DNA-polymerases such as Taq and typically requires 1-2 minutes per cycle to amplify 1 kb of DNA at 72° C. Also using Pfu DNA-polymerase in PCR reactions produces blunt-ended PCR products in contrast to Taq which produces dA overhang at the 3′-end.

### Table 1

TABLE 1

| DNA- | T_{1/2} at | Extension | Type of | |
|---|---|---|---|---|
| Polymerase | 95 C.°, min | rate nt/s | ends | Biological Source |
| Taq pol | 40 | 75 | 3′A | Thermus aquaticus |
| Amp litaq | 80 | >50 | 3′A | Thermus aquaticus |
| (Stoffel | | | | |
| fragment) | | | | |
| Vent* | 400 | 80 | 95% blunt | Thermococcus |
| | | | | litoralis |
| Deep Vent* | 1380 | NA | 95% blunt | Pyrococcus GB-D |
| Pfu* | >120 | 60 | Blunt | Pyrococcus furiosus |
| T th* | 20 | 30 | 3′A | Thermococcus |
| (RT activity) | | | | thermophilus |

*Have proof-reading function and can generate product over 30 kbp.

Have proof-reading function and can generate product over 30 kbp.

In contrast, Taq pol and its variant Amp litaq lack the 3′ to 5′ exonuclease proofreading activity resulting in relatively low replication fidelity. It has an estimated error rate at about 1 in 9,000 nucleotides, and thus, they are suitable for amplification of relatively short segments of less than 8000 bp or producing nucleic acid sequences having random mutations. One advantage of Taq DNA-polymerase is that it produces DNA products having dA overhangs at their 3′ ends. This may be useful in TA cloning, whereby a cloning vector (such as a plasmid) that has a dT 3′ overhang is used, which is complemented by the dA overhang of the PCR product, thus enabling ligation of the PCR product into the plasmid vector. Thus, Pfu DNA-polymerase is superior to Taq DNA-polymerase for techniques that require high-fidelity DNA synthesis. The combination of Pfu DNA-polymerase and Taq DNA-polymerase in one DNA-polymerase cocktail provides the advantages of both enzymes in PCR, i.e., the high fidelity of Pfu DNA-polymerase and the speed of Taq DNA-polymerase activity.

As used herein “one unit of DNA-polymerase activity” is defined as the amount of enzyme that incorporates 10 nmol of total deoxyribonucleoside triphosphates into acid perceptible DNA within 60 min at +65° C. The assay is carried out in 67 mM Tris/HCl; pH 8.3/25° C., 5 mM MgCl2, 10 mM mercaptoethanol, 0.2% polydocanol, 0.2 mg/ml gelatine, 0.2 mM each dATP, dGTP, dTTP and 0.1 mM dCTP in the presence of M13mp9ss as a template and M13 primer (17-mer), and 1 μCi (α³²P) dCTP. The produced DNA is precipitated by trichlroacetic acid, filtered, and quantified by scintillation counting. In some preferred embodiment of the method, the amount of DNA-polymerase added to a PCR reaction mixture is in the range of 0.1 to 3, preferably 0.2-2.5, preferably 0.5-2.0, preferably 0.7-1.5, preferably 1.0-1.25 units/50 μL of reaction mixture.

PCR Method Typically, PCR consists of a series of at least 10, preferably 20, preferably 30, preferably 40, preferably 50, preferably 60, preferably 70, preferably 80, preferably 100 or more repeated temperature changes, called thermal cycles. Each cycle consists of two or three discrete temperature steps. The cycling is often preceded by a single temperature step at a temperature of at least 90° C., preferably 95° C. for at least 5 s, preferably 10 s, preferably 15 s, preferably 20 s, preferably 25 s or more if needed. The temperatures used and the length of time they are applied in each cycle depend on a variety of parameters, including the enzyme used for DNA synthesis, the concentration of bivalent ions and dNTPs in the reaction, and the melting temperature (Tₘ) of the primed template. The individual steps common to most PCR methods are as follows:

Initialization: This step is only required for DNA polymerases that require heat activation by hot-start PCR. It consists of heating the reaction mixture at a temperature of 94-98° C. for at least 1 min, preferably 2 min, preferably 5 min, preferably 7 min, preferably 10 min or more if needed.

Denaturation: The first regular cycling event consists of heating the reaction mixture to at least 80° C., preferably 85° C., preferably 90° C., preferably 92° C., preferably 93° C., preferably 95° C., or preferably 96° C. for at least 5 s, preferably 10 s, preferably 15 s, preferably 20 s, preferably 25 s or more if needed to denature the double stranded DNA and separate the complementary strands..

Annealing: The annealing temperature is dependent on the length of the primer; longer primers would require higher annealing temperature than shorter primer and vice versa. Generally the annealing temperature in the range of 20-68° C., preferably 30-60° C., preferably 35-55° C., or preferably 40-50° C. for a time in the range of 10-60 s, preferably 15-45 s, preferably 20-40 s, preferably 20-30 s, allowing annealing of the primers to each of the single-stranded DNA templates. Two different primers are typically included in the reaction mixture: one for each of the two single-stranded complements containing the target region. It is critical to determine a proper temperature for the annealing step because efficiency and specificity are strongly affected by the annealing temperature. The temperature must be sufficiently low enough to allow for hybridization of the primer to the template, but high enough for the hybridization to be specific. A typical annealing temperature is about 3-5° C. below the Tₘ of the primed template used.

Extension/elongation: The temperature at this step depends on the DNA polymerase used which corresponds or near to the optimum activity temperature for the thermostable DNA polymerase. For example, Taq DNA-polymerase has optimal polymerization temperature in the range of 75-80° C., though a temperature of about 72° C. is commonly used. In an extension step, the DNA polymerase elongates the primer to a new DNA strand complementary to the DNA template by adding dNTPs from the reaction mixture that are complementary to the template in the 5′-to-3′ direction by forming a phosphodiester bond between the 3′-end of the primer and α-phosphate group of the 5′-nucleotide triphosphates. The time required for elongation depends on both the DNA polymerase used, its amount in the reaction mixture, and on the length of the DNA target region to be amplified. As a rule of thumb, at their optimal temperature, most thermostable DNA-polymerases polymerize a thousand bases in about 1-3 minutes. Under optimal conditions, at each extension/elongation step, the number of DNA target sequences is doubled. With each successive cycle, the original template strands plus all newly generated strands become template strands for the next round of elongation, leading to exponential (geometric) amplification of the specific DNA target region. The processes of denaturation, annealing and elongation constitute a single cycle. Multiple cycles are required to amplify the DNA target to a large number of copies. The formula used to calculate the number of DNA copies formed after a given number of cycles is 2ⁿ, where n is the number of cycles. Thus, a reaction set for 30 cycle's results in 2ⁿ, or 1,073,741,824, copies of the original double-stranded DNA target region.

Final elongation: This is an optional step is performed at a temperature in the range of 70-74° C. which is the temperature range required for optimal activity of most thermostable DNA-polymerases used in PCR for 5-15 minutes after the last PCR cycle to ensure that any remaining single-stranded DNA is fully elongated.

Final step: The reaction mixture is cooled down to a temperature in the range of 4-15° C. and stored or purified. In some embodiment of the invention, the double stranded DNA is isolated by well-known method in the art and the two strands are separated by denaturing electrophoreses since the complementary strand have different length. The modified primer at the 5′-end would produce a DNA comprising the modification at the 5′-end, whereas the other primer would produce only the complementary strand, which is the desired strand in some cases.

In some embodiments, the purified DNA strand modified at the 5′-end is used as a template in a PCR amplification reaction mixture comprising a forward primer with a free 3′-end and a reverse primer which does not contain extendable 3′-hydroxyl group. The non-extendable primers are well-known in the art and can be made by introducing a dideoxynucleotide or 3′-protected hydroxyl group at the 3′-end of primers. Dideoxynuclpotides derivative suitable for use in DNA-synthesis are commercially available from many vendors such as Sigma-Aldrich, USA. Amplification of such a PCR reaction mixture as described herein above would lead to the formation of only the complementary DNA strand, which may be separated from 5′-end modified template by denaturing gel electrophoreses or other means.

Thermal cycler, also known as a thermocycler, PCR machine, or DNA amplifier, is a laboratory apparatus most commonly used to amplify segments of DNA via PCR. The device has a thermal block with holes where tubes holding the reaction mixtures can be inserted. The cycler then raises and lowers the temperature of the block in discrete, pre-programmed steps. Several models having different capacity are commercially available from several venders; see for example Carolina® [https-://www.carolina.com/biotechnology-teaching-resources/polymerase-chain-reaction/pcr-equipment-supplies/], Sigma-Aldrich [https://www.sigmaaldrich.com/catalog/search?term=Labnet+MultiGene™+Gradient+PCR+The rmal+Cycler&interface=Product %20Na], Applied Biosystems, U. S. A, and ThermoFisher Scientific [https-://www.thermofisher.com/us/en/home/life-science/pcr/thermal-cyclers-realtime-instruments.html].

Several available methods well-known in the art for the processing of the PCR product may be utilized to purify and isolate the double stranded product. In some embodiments, the PCR product obtained with a modified primer contained an oligo- or poly-dA, -dT, -dG, or -dC may be isolated directly by a complementary sequence to the attached oligo or polynucleotide attached to the 5′-end of the modified primer. For example, the PCR product produce with the use of a modified primer with an oligo or poly-dA may be separated from the reaction mixture by an immobilized oligo- or poly-dT on a solid support followed by denaturation of the nucleic acid bound to the solid support in solution to separate the DNA from the solid support. Oligo (dT)₂₅ supported on magnetic beads is commercially available by the name Dynabeads™ Oligo(dT)₂₅ from ThermoFisher Scientific, USA. Then, the product DNA strands may be separated by denaturing the DNA followed by separation by well-known methods in the art such as, but not limited to gel or capillary electrophoreses, or ion exchange or reverse phase chromatography. In some other embodiments, the PCR reaction mixture is loaded directly on a gel to be separated. It may be desirable in some instances to treat the reaction product with alkaline phosphatase to remove nucleotide triphosphates. Also, it may be desirable in some instances to treat the product mixture of the PCR with a thermolabile exonuclease to digest the single stranded primers, but care should be taken in selecting the exonuclease. In some embodiments where the modified primer used in the PCR contains two 3′-ends (see FIG. 7), an exonuclease digesting a single stranded DNA in 5′-3′ direction such as beef spleen phosphodiesterase, available commercially from Sigma-Aldrich, U. S. A should be used. On the other hand, when a modified primer comprising 3′ and 5′-ends (see FIG. 8) is used, an exonuclease digesting a single stranded DNA in the 3′-5′ direction such as E. coli exonuclease I or snake venom phosphodiesterase, both available commercially from Sigma-Aldrich, U.S.A

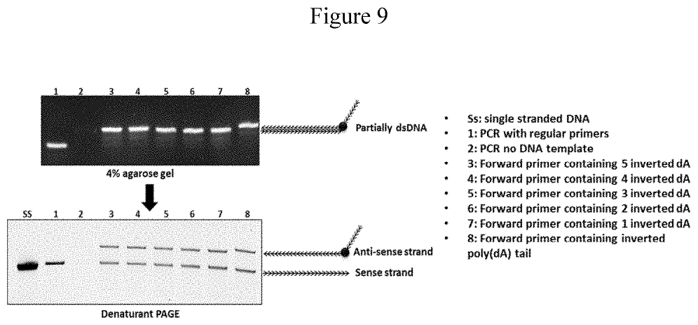

Gel electrophoresis is a method for separation and analysis of macromolecules such as DNA, RNA and proteins and their fragments, based on their size and charge. It is used to separate proteins by charge or size, and a mixed population of DNA or RNA fragments by length and charge, to estimate the size of DNA or RNA fragments. Nucleic acid molecules are separated by applying an electric field to move the negatively charged molecules through a matrix of agarose or other substances. Shorter molecules move faster and migrate farther than longer ones because shorter molecules migrate more easily through the pores of the gel. This phenomenon is called sieving. Gel electrophoresis utilizes a gel as an anti-convective medium or sieving medium during electrophoresis, the movement of a charged particle in an electrical field. The gel suppresses the thermal convection caused by application of the electric field. Also, it acts as a sieving medium, retarding the passage of molecules and maintains the finished separation. DNA Gel electrophoresis is usually performed for analytical or preparative purposes. It is used after amplification of DNA via PCR to separate different fragments of DNA. As used herein, electrophoresis is a process which enables the sorting of molecules based on size. Using an electric field, DNA molecules can be made to move through a gel made of agarose or polyacrylamide. The electric field consists of a negative charge at one end which pushes the molecules through the gel, and a positive charge at the other end that pulls the molecules through the gel. The molecules being sorted are deposited in a well in the gel material. The gel is placed in an electrophoresis chamber, which is then connected to a power source. When the electric current is applied, the larger molecules move more slowly through the gel while the smaller molecules move faster. The different sized molecules form distinct bands on the gel. The term “gel” refers to a matrix used to contain and separate the DNA molecules. In most cases, the gel is a crosslinked polymer whose composition and porosity is chosen based on the specific weight and composition of the target to be analyzed. When small nucleic acids of DNA, RNA, or oligonucleotides, the gel is usually composed of different concentrations of acrylamide and a cross-linker, producing different sized mesh networks of polyacrylamide. When separating larger nucleic acids, greater than a few hundred bases, the preferred matrix is purified agarose. In both cases, the gel forms a solid, yet porous matrix. Acrylamide, in contrast to polyacrylamide, is a neurotoxin and must be handled with care using appropriate safety precautions to avoid poisoning. Agarose is composed of long unbranched chains of uncharged polysaccharide without cross links resulting in a gel with large pores allowing for the separation of macromolecules and macromolecular complexes. If several samples have been loaded into adjacent wells in the gel, they will run parallel in individual lanes. Depending on the number of different molecules, each lane shows separation of the components from the original mixture as one or more distinct bands, one band per component. Incomplete separation of the components can lead to overlapping bands, or to indistinguishable smears representing multiple unresolved components. Bands in different lanes that end up at the same distance from the top contain molecules that passed through the gel with the same speed, which usually means they are approximately the same size, see for example FIG. 9. There are molecular weight size markers available that contain a mixture of molecules of known sizes. If such a marker was run on one lane in the gel parallel to the unknown samples, the bands observed can be compared to those of the unknown in order to determine their size. The distance a band travels is approximately inversely proportional to the logarithm of the size of the molecule.

The types of gel most typically used are agarose and polyacrylamide gels. Each type of gel is well-suited to different types and sizes of analyte. Polyacrylamide gels are usually used for proteins, and have very high resolving power for small fragments of DNA (5-500 bp). Agarose gels on the other hand have lower resolving power for DNA but have greater range of separation, and are therefore used for DNA fragments of usually 50-20,000 bp in size, and resolution of over 6 Mb is possible with pulsed field gel electrophoresis (PFGE). Polyacrylamide gels are run in a vertical configuration while agarose gels are typically run horizontally in a submarine mode. Also they differ in their casting methodology, as agarose sets thermally, while polyacrylamide gel forms in a chemical polymerization reaction. For the separation or resolution of large 5-10 kb DNA fragments, agarose gels are made of about 0.7% agarose dissolved in electrophoresis buffer. In contrast for small nucleic acid fragment of 200-1000 bp, 2% agarose gel provides good resolution. For smaller fragments of nucleic acids, up to 3% agarose or polyacrylamide may be used. Low percentage gels are very weak and may be fragmented when they are handled and high percentage gels are often brittle and do not set evenly. For most common application, 1% agarose gel is used. Gel electrophoreses equipment and supplies including power supplies, agarose, acrylamides, buffers, pre-casted agarose and polyacrylamide gels are commercially available from laboratory supply venders such as Sigma-Aldrich, USA, Biorad, USA, and Thermo-Fisher, USA.

The two strands obtained from the PCR method of the invention are of different length, and therefore, can be separated by denaturing gel electrophorese. Once the gel is developed (FIG. 9), the desired bands are cut and suspended in an appropriate buffer to release the single stranded DNA.

In some embodiments, it may be desirable to provide an additional or alternative means for separating the two nucleic acid strands produced by the method of the invention. Capillary electrophoresis (CE) is a family of electrokinetic separation methods performed in submillimeter diameter capillaries and in micro- and nanofluidic channels. Very often, CE refers to capillary zone electrophoresis (CZE), but other electrophoretic techniques including capillary gel electrophoresis (CGE), capillary isoelectric focusing (CIEF), capillary isotachophoresis and micellar electrokinetic chromatography (MEKC) belong also to this class of methods. In CE methods, analytes migrate through electrolyte solutions under the influence of an electric field. Analytes can be separated according to ionic mobility and/or partitioning into an alternate phase via non-covalent interactions. Additionally, analytes may be concentrated or “focused” by means of gradients in conductivity and pH. Electrophoresis of a DNA sample through the capillary provides a size based separation profile for the sample. Microcapillary array electrophoresis generally provides a rapid method for size-based sequencing, PCR product analysis, and restriction fragment sizing. The high surface to volume ratio of these capillaries allows for the application of higher electric fields across the capillary without substantial thermal variation across the capillary, consequently allowing for more rapid separations. Furthermore, when combined with confocal imaging methods, these methods provide sensitivity in the range of attomoles, which is comparable to the sensitivity of radioactive sequencing methods.

Capillary electrophoreses instruments are commercially available from various vendors. Alternatively, microcapillary electrophoretic devices has been discussed in detail in, for example, Jacobson et al., Anal Chem, 66:1107-1113, 1994; Effenhauser et al., Anal Chem, 66:2949-2953, 1994; Harrison et al., Science, 261:895-897, 1993; Effenhauser et al., Anal Chem, 65:2637-2642, 1993; Manz et al., J. Chromatogr 593:253-258, 1992; and U.S. Pat. No. 5,904,824—incorporated herein by reference. Typically, these methods comprise photolithographic etching of micron scale channels on a silica, silicon, or other crystalline substrate or chip, and can be readily adapted for use in the present disclosure.

Tsuda et al. (Anal Chem, 62:2149-2152, 1990) describes rectangular capillaries, an alternative to the cylindrical capillary glass tubes. Some advantages of these systems are their efficient heat dissipation due to the large height-to-width ratio and, hence, their high surface-to-volume ratio and their high detection sensitivity for optical on-column detection modes. These flat separation channels have the ability to perform two-dimensional separations, with one force being applied across the separation channel, and with the sample zones detected by the use of a multi-channel array detector.

In many capillary electrophoresis methods, the capillaries, e.g., fused silica capillaries or channels etched, machined, or molded into planar substrates, are filled with an appropriate separation/sieving matrix. Typically, a variety of sieving matrices known in the art may be used in the microcapillary arrays. Examples of such matrices include, e.g., hydroxyethyl cellulose, polyacrylamide, agarose, and the like. Generally, the specific gel matrix, running buffers, and running conditions are selected to maximize the separation characteristics of the particular application, e.g., the size of the nucleic acid fragments, the required resolution, and the presence of native or undenatured nucleic acid molecules. For example, running buffers may include denaturants, chaotropic agents such as urea to denature nucleic acids in the sample.

Since the two DNA strands produced by the method of invention are of different size and charges, they may be separated by denaturing capillary electrophoreses. DNA separations are carried out using thin CE, 50-mm, fused silica capillaries filled with a sieving buffer. These capillaries have excellent capabilities to dissipate heat, permitting much higher electric field strengths to be used than slab gel electrophoresis. Therefore separations in capillaries are rapid and efficient. Additionally, the capillaries can be easily refilled and changed for efficient and automated injections. Detection occurs via fluorescence through a window etched in the capillary. Both single-capillary and capillary-array instruments are available with array systems capable of running 16 or more samples simultaneously for increased throughput.

In some other embodiments, the PCR DNA product strands may be separated on an ion exchange column run under denaturing condition. Many ion exchange columns are commercially available. In a preferred embodiment, either a string or weak cation exchange column may be utilized for the separation.

### Example 1

### Modified Primers:

Six reverse primers modified at the 5′-ends were prepared as described herein. Each primer contained at its 3′ end 24 nt, complementary to the ssDNA template, and at its 5′end an extra poly-dA tail of 22 nt in length. In one of the 6 tested forward primers, the poly-dA was constituted completely of 22 internally inverted dAs. In this case the inverted poly-dA tail was attached to the forward primer by a 5′-5′ linkage (FIG. 5). In the other set of primers the poly-dA tail was attached to the 3′ region by either one, two, three, four or five, internally inverted dA. The internally inverted dAs are linked to their flanking nucleotides by 3′-3′ and 5′-5′ linkages (FIG. 6).

### PCR

It is well established that DNA polymerase requires a free 3′OH group for initiation of synthesis and thus can synthesize DNA in only one direction by extending the 3′end of a pre-existing primer moving along the template strand in a 3′-5′ direction. This property of DNA polymerase was exploited to investigate whether incorporation of internally inverted nucleotide/s, at the 5′ end of the ssDNA template, through 3′-3′/5′-5′ linkages could be used as a new strategy to stop DNA polymerization in PCR. Using this strategy, the PCR product will include two complementary DNA strands having different lengths and separable from one another. This type of PCR strategy will be an efficient way for ssDNA amplification.

The efficiency of internally inverted nucleotides as a terminator of DNA polymerization during PCRs was assessed. Six reverse primers with different lengths (numbers) of inverted dAs were used. A reverse primer without inverted dA or a polyA tail was used as a control.

Standard PCR mixtures of 50 μL each were prepared comprising 0.25 mM of one of the modified reverse primer (composed of 24 complementary nt and a poly-dA tail of 26 nt; Tm: 67° C.), 0.25 mM regular forward primer (24 nt in length; Tm: 65° C.), 0.2 mM each of dATP, dCTP, dGTP, and dTTP, 50 mM KCl, 10 mM Tris buffer pH 8.4, 1.5 mM MgCl₂, about 0.003 units of Taq polymerase, and 1 mL from a solution (0.1 mM) of the ssDNA template (100 nt). The thermocycling program used started with 1 cycle at 95° C. for 5 min, followed by 30 cycles of 95° C. for 30 sec, 65° C. for 30 sec, and 72° C. for 30 sec. Parts (10 ml) of each reaction product mixture were loaded on a multi wells 4% native agarose gel. Another fraction from purified PCR product (half of total PCR product) was analyzed by denaturing PAGE. The results are shown in FIG. 9. PCR product prepared with regular primer displayed only one band on the gel (lane 1 of FIG. 9). In contrast, the PCR products from reaction mixture using modified reverse primers were resolved into two clear bands (lines 3-8 of FIG. 9). This indicates that incorporation of one or more internally inverted dA was strong enough to terminate DNA polymerization, resulting in dsDNA product with 2 complementary strands of different length. All DNA-oligonucleotides used in this experiment were purchased from Integrated DNA Technologies (IDT, USA; www.idtdna.com).

1: A PCR method for the synthesis of a single stranded nucleic acid sequence, comprising:
preparing a polymerase chain reaction comprising a template, a first modified primer, a second primer, a nucleotide triphosphate, and a DNA-polymerase,
carrying out a plurality of amplification reactions by cycling the temperature of the reaction mixture between 20° C. and 100° C. to form product DNA strands, and
isolating the product DNA strands;
wherein the first modified primer comprises a first oligonucleotide segment of 10-50 nucleotides in length complementary to a target nucleic acid and having an extendable 3′-end and a 5′-end linked to the 5′-end of a second oligonucleotide segment of 10-100 nucleotides or a polynucleotide.

2: The method of claim 1, wherein the product DNA strands are isolated by denaturing electrophoresis.

3: The method of claim 1, wherein the template is a DNA strand.

4: The method of claim 3, wherein the DNA-polymerase is a thermophilic DNA-dependent DNA polymerase.

5: The method of claim 1, wherein the second oligonucleotide segment is an oligo-dA, or poly-dA.

6: The method of claim 5, further comprising:
separating the product DNA strands from the reaction mixture by binding to oligo- or poly(dT) or (U) supported on a solid support.

7: The method of claim 6, further comprising:
releasing the product DNA strands from the solid support by denaturation.

8: The method of claim 1, wherein the template is a RNA strand.

9: The method of claim 8, wherein the DNA polymerase is an RNA-dependent DNA-polymerase.

10: A PCR method for the synthesis of a single stranded nucleic acid sequence, comprising:
preparing a polymerase chain reaction comprising a template, a first modified primer, a second primer, a nucleotide triphosphate, and a nucleic acid-polymerase,
carrying out a plurality of amplification reactions by cycling the temperature of the polymerase chain reaction between 20° C. and 100° C. to form a reaction mixture comprising product DNA strands, and
isolating the product DNA strands;
wherein the modified primer comprises a first oligonucleotide segment of 10-50 nucleotides complementary to a target DNA template having an extendable 3′-end and a 5′-end linked to the 5′-end of a nucleotide or dinucleotide which is linked through its 3′-end to a 3′-end through 3′-3′ linkage to a second oligonucleotide segment of 10-100 nucleotides or a polynucleotide.

11: The method of claim 10, wherein the product DNA strands are isolated by denaturing electrophoresis.

12: The method of claim 10, wherein the template is a DNA strand.

13: The method of claim 10, wherein the DNA polymerase is a thermophilic DNA-dependent DNA polymerase.

14: The method of claim 10, wherein the second oligonucleotide segment is linked to oligo-dA, or poly-dA.

15: The method of claim 14, wherein the DNA strands are removed from the reaction mixture by a solid support modified by a fragment of oligo or poly(dT) or (U).

16: The method of claim 15, wherein the product DNA strands are isolated by denaturing electrophoresis.

17: The method of claim 10, wherein the template is a RNA strand.

18: The method of claim 8, wherein the DNA polymerase is RNA-dependent DNA polymerase.

19: A polynucleotide primer, comprising:
a first oligonucleotide segment and a second oligonucleotide segment linked by 5′-5′-phosphodiester bond,
wherein the first oligonucleotide segment comprises a 10-50 nucleotide sequence complementary to a target nucleic acid for amplification and the second oligonucleotide segment is an oligonucleotide of 10-99 residues or a polynucleotide.

20: The polynucleotide primer of claim 20, wherein the second oligonucleotide segment is oligo- or poly-dA, oligo- or poly-dT, oligo- or poly-dG, or oligo- or poly-dC.
